(19) United States
(12) Patent Application Publication
(10) Pub. No.: US 2021/0214676 A1
(43) Pub. Date: Jul. 15, 2021

(54) COMPOSITION

(71) Applicant: 4D Pharma Research Limited, Aberdeen (GB)

(72) Inventors: Samantha YUILLE, Aberdeen (GB); Nicole REICHARDT, Aberdeen (GB)

(21) Appl. No.: 17/089,370

(22) Filed: Nov. 4, 2020

Related U.S. Application Data
(63) Continuation of application No. PCT/EP2019/061446, filed on May 3, 2019.

(30) Foreign Application Priority Data
May 4, 2018 (EP) 18170880.1

(51) Int. Cl. C12N 1/20; C12Q 1/02; G01N 33/50

(52) U.S. Cl. CPC C12N 1/20; G01N 33/5038; C12Q 1/02

(57) ABSTRACT

The invention provides compositions, processes and kits for determining the metabolic properties of cells of interest in vitro.

### CROSS-REFERENCE

This application is a continuation of International Application No. PCT/EP2019/061446, filed May 3, 2019, which claims the benefit of European Application No. 18170880.1, filed May 4, 2018, all of which are hereby incorporated by reference in their entirety.

### TECHNICAL FIELD

The present invention provides a simulated intestinal environment which permits the metabolic properties of cells of interest to be determined in vitro. The invention also relates to systems employing such environments and processes in which such environments and systems are utilised.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like. The human gut microbiota contains more than 500-1000 different phylotypes belonging essentially to two major bacterial divisions, the Bacteroidetes and the Firmicutes.

The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized.

Given the importance of these symbiotic relationships on the maintenance of health, there is significant interest in identifying the effects of specific cells (e.g. bacterial or fungal cells) on the operation of the microbiome, whether those cells are considered to be beneficial to health or pathogenic. It is also important to verify that the properties of a cell of interest observed in a simple in vitro assay will still be imparted in the significantly more complex intestinal environment.

In vivo studies can be used to investigate the effects of cells of interest. Animal models are a convenient way to enable the effects of specific cells to be assessed. The animals used in such studies may be healthy or may be manipulated or challenged to exhibit the symptoms of a specific disease and changes in behaviour or disease state imparted by the cells of interest can be observed. Additionally, the effects of the cells of interest can also be determined via other means, for example by fecal analysis (to see whether administration of the cells under investigation caused any changes or perturbations on the composition of the microbiome) and/or metabolomic analysis (to see whether administration of the cells resulted in any changes in the profile of metabolites either in the GI tract or in distal tissue).

One issue with animal studies, however, is that because they are carried out in a living organism influenced by innumerable and complex biological reactions, it is not always possible to conclusively determine that a physiological change observed following administration of the cells of interest necessarily arises as a result of that administration or for some other reason.

Additional factors which can influence the outcome and reliability of the results of such studies include genetic differences and inconsistent environmental conditions between the animals which are employed. While it is possible to minimise the impact of these differences (e.g. by control of breeding conditions and genetic screening of the animals and/or by ensuring consistency in storage and feeding of the animals), these steps add to the cost and complexity of studies, especially where carried out in multiple sites.

A further issue with animal studies is that there are numerous steps that must be taken from the commencement of a study design to the generation of meaningful data which can mean that this can take several weeks if not months.

As an alternative approach to animal models, attempts have been made to develop simulated gastrointestinal environments. For example, the Simulator of the Human Intestinal Microbial Ecosystem)(SHIME®), discussed on pages 305 to 317 of The Impact of Food Bioactives on Health (2015) is a simulator of the human gut in which a series of fermenters are operated to mimic various regions of the GI tract including the stomach, small intestine, and different regions of the colon.

In order to mimic the gut, the developers of the SHIME® system sought to include inoculum in each fermenter representative of colonic regions of the GI tract which was as close to that existing in human subjects as possible. To most accurately reflect the in vivo situation, material derived from fecal samples is used. However, the aim of mimicking the ‘normal’ microbiome is not straightforward as across even a small number of subjects, the composition of the microbiome will vary substantially. Even the microbiome composition in a single subject will fluctuate substantially over time. Accordingly, owing to the complexity of the bacterial composition as well as the inter-sample variability, it is challenging to repeatably conclude with certainty that an observed change following administration of an agent of interest was caused by that administration or by some other variation in the microbial inoculum.

As explained in the above-mentioned chapter from The Impact of Food Bioactives on Health, there has been debate as to whether the inoculum used in the SHIME® system should be derived from a single donor or from a pool of donors and there are advantages and drawbacks of both approaches.

It has also been reported in that chapter that, in order to obtain stable functionality in terms of short chain fatty acid production in the SHIME® system, an adaptation period of at least 15 if not 20 days was needed. Only once stability of metabolite production has been achieved can the test substance be introduced into the inoculum.

Another in vitro model for the gut microbiome referred to as TIM-2 by Venema (2015, The Impact of Food Bioactives on Health, pages 293 to 304) has a shorter set-up time, but again uses fecal inoculate from volunteers which again is problematic owing to sourcing of such samples, inter-donor variability and safety concerns due to pathogens within such inoculate.

The human microbiota is known to contain at least 1000 different species of bacteria (Lozupone et al. (2012) Nature 489 (7415) 220-230). The microbiome is highly variable between individuals and can change in response age, diet, disease and the environment. Samples obtained from one subject over time are more similar than those obtained from a different subject, which suggests that each person has a relatively distinct microbiome. The TIM-2 and SHIME systems are inoculated with human faecal samples. Therefore, the bacterial populations used to inoculate these systems are highly variable populations, which can for example lead to major differences in metabolite production between the systems.

There is therefore a need in the art for a more controlled simulated gastrointestinal environment which addresses one or more of the shortcomings of the prior art outlined above.

### SUMMARY OF THE INVENTION

Thus, according to a first aspect of the present invention, there is provided a simulated intestinal environment comprising a liquid medium, a nutritional energy source and a bacterial population consisting essentially of 5 to 200 species of bacteria wherein said bacterial population is capable of producing at least three of butyrate, propionate, succinate, ethanol, acetate, lactate and formate from said nutritional energy source.

Advantageously, the simulated intestinal environment permits the metabolic interactions of cells to be determined in a simpler, more controlled and more repeatable manner than with the systems of the prior art. Unexpectedly, the inventors have found that accurate predictions of in vivo metabolic activity can be made in a simulated intestinal environment comprising far fewer species of organisms than were conventionally thought to be required for such assessments to be made. Those skilled in the art will recognise that a bacterial population comprising 200 or fewer different species is significantly less complex and more controlled than material derived from a fecal sample which will contain many thousands of different species of bacteria.

Indeed, as demonstrated in the examples which follow, the inventors have demonstrated that a simulated intestinal environment comprising a bacterial population consisting essentially of fewer than 200 bacterial species can be used to provide valuable insights into the metabolic activity of cells of interest. In particular, a bacterial population consisting essentially of fewer than 200 bacterial species can be useful in providing valuable insights into the metabolic activity of cells of interest. In preferred embodiments of the invention the bacterial population consists essentially of 6 or more, 7 or more, 8 or more, 9 or more, 10 or more, 11 or more, 12 or more, 13 or more, 14 or more, 15 or more or 16 or more species of bacteria. Additionally or alternatively, the bacterial population may consist essentially of 150 or fewer, 120 or fewer, 100 or fewer, 90 or fewer, 80 or fewer, 70 or fewer, 60 or fewer, 50 or fewer, 40 or fewer, 30 or fewer, 25 or fewer or 20 or fewer species of bacteria. In a preferred embodiment, the bacterial population consists essentially of 5 to 50 species of bacteria. In a more preferred embodiment, the bacterial population consists essentially of 10 to 30 species of bacteria. In further preferred embodiments, the bacterial population consists of essentially 10 to 20 species of bacteria.

In some embodiments of the invention, the bacterial population does not contain more than 200 species of bacteria. In preferred embodiments, the simulated intestinal environment is not a SHIME or TIM-2 system. In preferred embodiments, the bacterial population is not inoculated with a faecal sample, such as a human faecal sample.

It is understood that the simulated intestinal environment will generally be an in vitro simulated intestinal environment.

In some embodiments of the invention, the bacterial population consists essentially of between 5 and 200 different strains of bacteria. In some embodiments, the bacterial population consists essentially of 6 or more, 7 or more, 8 or more, 9 or more, 10 or more, 11 or more, 12 or more, 13 or more, 14 or more, 15 or more or 16 or more strains of bacteria. Additionally or alternatively, the bacterial population may consist essentially of 150 or fewer, 120 or fewer, 100 or fewer, 90 or fewer, 80 or fewer, 70 or fewer, 60 or fewer, 50 or fewer, 40 or fewer, 30 or fewer or 25 or fewer strains of bacteria. In a preferred embodiment, the bacterial population consists essentially of 5 to 50 strains of bacteria. In a more preferred embodiment, the bacterial population consists essentially of 10 to 30 strains of bacteria. In further preferred embodiments, the bacterial population consists of essentially 10 to 20 strains of bacteria. The bacterial population may consist essentially of 5 to 50 strains of bacteria. In a more preferred embodiment, the bacterial population consists essentially of 10 to 30 strains of bacteria. In further preferred embodiments, the bacterial population consists essentially of 10 to 20 strains of bacteria.

The bacterial population may include one or more bacterial species from families selected from the group consisting of: Bacteroidaceae, Prevotellaceae, Rikenellaceae, Ruminococcaceae, Veillonellaceae and/or Verrucomicrobiaceae. The bacterial population can include species from the genera Alistipes, Akkermansia, Anaerostipes, Bacteroides Bacteroidetes, Bariatricus, Bifidobacterium, Blautia, Butyrivibrio, Butyricicoccus, Coprococcus, Clostridium, Collinsella, Dialister, Desulfovibrio, Dorea, Escherichia, Eubacterium, Firmicutes, Faecalibacterium, Lactobacillus, Megasphaera, Methanobrevibacter, Phascolarctobacterium, Prevotella, Ruminococcus, Roseburia, Selenomonas and/or Verrucomicrobia.

The bacterial population of the invention may include one or more bacterial species selected from the group consisting of: Alistipes putredinis, Akkermansia muciniphila, Anaerostipes caccae, Anaerostipes coli, Anaerostipes hadrus, Anaerostipes rhamnosivorans, Bacteroides coccoides, Bacteroides dorei, Bacteroides massiliensis, Bacteroides thetaiotamicron, Bacteroides uniform is, Bacteroides vulgatus, Bariatricus massiliensis, Bifidobacterium adolescentis, Bifidobacterium longum, Blautia producta, Blautia coccoides, Blautia obeum, Butyricicoccus pullicaecorum, Butyrivibrio fibrisolvens, Clostridium butyricum, Clostridium indolis, Clostridium innocuum, Clostridium propionicum Coprococcus catus, Coprococcus comes, Coprococcus eutactus, Dialister invisus, Desulfovibrio piger, Dorea longicatena, Escherichia coli, Eubacterium cylindroides, Eubacterium hallii, Eubacterium limosum, Eubacterium ramulus, Eubacterium rectale, Faecalibacterium prausnitzii, Lactobacillus salivarius, Megasphaera elsdenii, Methanobrevibacter smithii, Phascolarctobacterium succinatutens, Prevotella copri, Ruminococcus albus, Roseburia faecis, Roseburia hominis, Roseburia intestinalis, Roseburia inulinivorans, Selenomonas ruminantium and/or Veillonella parvula.

In more preferred embodiments, the bacterial population consists of the following species: Bacteroides dorei, Bacteroides uniformis, Bacteroides thetaiotaomicron, Bacteroides vulgatus, Bifidobacterium longum, Blautia producta, Blautia sp., Bariatricus massiliensis, Clostridium innocuum, Dorea longicatena, Eubacterium hallii, Escherichia coli, Eubacterium rectale, Lactobacillus salivarus, Prevotella sp. and Roseburia faecis. A bacterial population containing these species encompasses all the major metabolic pathways for the short chain fatty acids, which has been identified by the inventors as being of important in assessing the metabolic activity of cells of interest.

### BRIEF DESCRIPTION OF DRAWINGS

FIG. 1: Design of bacterial population.

FIG. 2: Schematic illustration of apparatus of the simulated intestinal environment.

FIG. 3: The levels of butyrate, propionate, acetate and formate assessed upon the addition of the bacterial population to the simulated intestinal environment.

FIG. 4: The Megasphaera massiliensis strain MRx0029 is a butyrate producing bacterial strain that can also produce valeric acid and the medium chain fatty acid (MCFA) hexanoic acid.

FIG. 5: Acetic acid, formic acid, propionic acid, butanoic acid, hexanoic acid produced by SimMi with added MRx0029 vs. SimMi at d1, d2, d3, d4, d5, d6, d7, d8, d9, d10, d11, d12, d13, and d14.

FIG. 6: Acetate, butyrate, hexanoic acid, and propionate produced by SimMi including MRx0029 vs. SimMi control at day 1, day 2, day 3, day 4, day 5, day 6, day 7, and day 8.

FIG. 7: Acetate, propionate, butyrate, valeric acid, and hexanoic acid produced by SimMi including MRx0029 vs. SimMi at day 11 and day 12.

FIG. 8: Total HDAC inhibition by aliquots from the SimMi and SimMi+MRX0029 cultures on whole HT-29 cells and HT-29 cell lysate.

### DISCLOSURE OF THE INVENTION

As used herein, to describe the bacterial population comprised within the simulated intestinal environment of the invention, the term “consisting essentially of” and ‘consisting of’ is used to characterise the simulated intestinal environment as excluding additional bacterial strains or species, or comprising only de minimis or biologically irrelevant amounts of other bacterial strains or species.

The major short chain fatty acid products in the gut microbiota include formate, acetate, propionate, lactate and butyrate (Douglas and Preston (2016) Gut Microbes, 7, (3), 189-200.) As mentioned above, the bacterial population is capable of producing at least three of butyrate, propionate, succinate, ethanol, acetate, lactate and formate from the nutritional energy source. In preferred embodiments, the bacterial population is capable of producing at least four of, at least five of, at least six of, or all of butyrate, propionate, succinate, ethanol, acetate, lactate and formate. A bacterial population capable of producing at least four of, at least five of, at least six of or all of butyrate, propionate, succinate, ethanol, acetate, lactate and formate encompasses the major metabolic pathways for the short chain fatty acids. This allows the bacterial population to accurately mimic the intestinal environment, which the inventors have identified as being of importance in assessing the metabolic activity of cells of interest.

For the avoidance of doubt, this does not necessarily mean that butyrate, propionate, succinate, ethanol, acetate, lactate and/or formate must be produced directly by the members of the bacterial population; those compounds may be indirectly produced. For example, a first bacterial strain may produce a first metabolite (e.g. lactate or succinate) from the nutritional energy source, and a second bacterial strain present in the bacterial population may produce a short chain fatty acid (e.g. propionate, butyrate, acetate and/or formate) from that first metabolite.

Additionally, it is not essential that each of butyrate, propionate, succinate, ethanol, acetate, lactate and/or formate are produced as end products in the simulated intestinal environment such that measurable levels of each of those compounds will be present in the simulated intestinal environment once stabilisation has been attained. For example, one or more of those compounds (e.g. succinate and/or acetate) may be produced as first metabolites by one or more strains in the bacterial population, which may then be converted by further members of the bacterial population into short chain fatty acids (e.g. propionate, butyrate, acetate and/or formate).

In embodiments of the invention, the simulated intestinal environment comprises one or more short chain fatty acids. In preferred embodiments the simulated intestinal environment comprises at least three of, at least four of or all five of butyrate, propionate, acetate, lactate and formate.

Some or all of these components, where present, may be produced by the bacterial population. Additionally or alternatively, some or all of these components may be added directly to the liquid medium.

For example, in some embodiments, butyrate, lactate and/or formate may be produced by the bacterial population. Additionally or alternatively, propionate and/or acetate may be added directly to the liquid medium. In some embodiments, acetate and propionate (if present) may be both added directly to the liquid medium and produced by the bacterial population.

In preferred embodiments, at least 1, at least 2, at least 3, at least 4, at least 5, at least 6 or at least 7 of butyrate, propionate, succinate, ethanol, acetate, lactate and formate are produced by the bacterial population. The inventors have found that it is advantageous to have the short chain fatty acids produced by the bacterial populations as this mimics the intestinal environment.

In embodiments in which the simulated intestinal environment comprises butyrate, butyrate is present at a concentration of at least about 0.5 mM, about 1.0 mM, about 1.5 mM, about 2.0 mM, about 3.0 mM, about 4.0 mM or about 5.0 mM. Additionally or alternatively, butyrate may be present at a concentration of about 10.0 mM or lower, about 8.0 mM or lower, about 6.0 mM or lower, or about 5.0 mM or lower. In a preferred embodiment in which the simulated intestinal environment comprises butyrate, butyrate is present at a concentration of about 1.0 mM to about 5.0 mM.

In embodiments in which the simulated intestinal environment comprises propionate, propionate is present at a concentration of at least about 0.5 mM, about 1.0 mM, about 1.5 mM, about 2.0 mM, about 3.0 mM, about 4.0 mM or about 5.0 mM. Additionally or alternatively, propionate may be present at a concentration of about 10.0 mM or lower, about 8.0 mM or lower, about 6.0 mM or lower, or about 5.0 mM or lower. In a preferred embodiment in which the simulated intestinal environment comprises propionate, propionate is present at a concentration of about 2.0 mM to about 7.0 mM.

In embodiments in which the simulated intestinal environment comprises acetate, acetate is present at a concentration of at least about 0.1 mM, about 0.2 mM, about 0.5 mM, about 1.0 mM, about 1.5 mM, or about 2.0 mM. Additionally or alternatively, acetate may be present at a concentration of about 5.0 mM or lower, about 4.0 mM or lower, about 3.0 mM or lower, or about 2.0 mM or lower. In a preferred embodiment in which the simulated intestinal environment comprises acetate, acetate is present at a concentration of about 0.2 to about 2.0 mM.

In embodiments in which the simulated intestinal environment comprises lactate, lactate is present at a concentration of at least about 0.1 mM, about 0.2 mM, about 0.5 mM, about 1.0 mM, about 1.5 mM, or about 2.0 mM. Additionally or alternatively, lactate may be present at a concentration of about 5.0 mM or lower, about 4.0 mM or lower, about 3.0 mM or lower, or about 2.0 mM or lower. In a preferred embodiment in which the simulated intestinal environment comprises lactate, lactate is present at a concentration of about 0.2 to about 2.0 mM.

In embodiments in which the simulated intestinal environment comprises formate, formate is present at a concentration of at least about 0.01 mM, about 0.02 mM, about 0.05 mM, about 0.1 M, about 0.2 mM, or about 0.5 mM. Additionally or alternatively, formate may be present at a concentration of about 2.0 mM or lower, about 1.5 mM or lower, or about 1.0 mM or lower. In a preferred embodiment in which the simulated intestinal environment comprises formate, formate is present at a concentration of about 0.01 to about 0.5 mM.

Where concentrations of components of the simulated intestinal environment are quantified herein, these are provided as molar amounts of the component in question in the liquid medium unless otherwise specified. Means for measuring these compounds are well known in the art and include liquid chromatography, HP-LC, LC-MS and gas chromatography. As shown in the examples quantification of components of the simulated intestinal environment can be done using gas chromatography.

In embodiments in which the simulated intestinal environment comprises at least three of, at least four of or all five of butyrate, propionate, acetate, lactate and formate, propionate or butyrate may be present at the highest concentration. Additionally or alternatively, in such embodiments, acetate, formate or lactate may be present at the lowest concentration.

Regarding the composition of the bacterial population, those skilled in the art will be familiar with bacteria capable of producing butyrate, propionate, succinate, ethanol, acetate, lactate and/or formate. Any such bacteria may be comprised within the bacterial population.

In embodiments, where butyrate producing bacteria are included in the bacterial population, these may produce butyrate via one or more of the following intermediate metabolites: phosphoenolpyruvate, pyruvate, acetyl CoA, acetoacetyl CoA, butyryl CoA, butyryl phosphate.

Human gut bacteria are known to produce propionate via three different pathways: 1) the succinate pathway; 2) the acrylate pathway; and 3) the propanediol pathway (Reichardt et al. (2014) ISME J. 8(6): 1323-1335). In certain embodiments of the invention the bacteria included in the bacterial population can produce propionate via one, two or all three pathways. In embodiments, where propionate producing bacteria are included in the bacterial population, these may produce propionate via one or more of the following intermediate metabolites: dihydroxyacetone phosphate, L-lactaldehyde, propane-1,2-diol, propionyl CoA, PEP, oxaloacetate, succinate, succinyl CoA, propionyl CoA, pyruvate, lactate or lactoyl CoA.

In certain embodiments, propionate produced via the succinate pathway can be produced by bacteria from the genera Bacteroides and/or Prevotella. In preferred embodiments, the propionate produced via the succinate pathway can be produced by any of the following species: Bacteroides dorei Bacteroides vulgatus Bacteroides uniformis, Bacteroides thetaiotaomicron and/or Prevotella copri.

In certain embodiments, propionate produced via the acrylate pathway can be produced by bacteria from the genera Eubacterium, Anaerostipes, Bifidobacterium and/or Lactobacillus. In preferred embodiments, the propionate produced via the acrylate pathway can be produced by any of the following species: Eubacterium hallii, Anaerostipes hadrus, Bifidobacterium longum and/or Lactobacillus salivarius.

In embodiments, where succinate producing bacteria are included in the bacterial population, these may produce succinate via one or more of the following intermediate metabolites: phosphoenolpyruvate, oxaloacetate.

In certain embodiments, propionate produced via the propanediol pathway can be produced by bacteria from the genera Blautia and/or Eubacterium. In preferred embodiments, the propionate produced via the propanediol pathway can be produced by Blautia obeum and/or Eubacterium hallii.

In embodiments, where ethanol producing bacteria are included in the bacterial population, these may produce ethanol via one or more of the following intermediate metabolites: phosphoenolpyruvate, pyruvate, acetyl CoA, acetaldehyde. In preferred embodiments, the ethanol producing bacteria in the bacterial population is Lactobacillus salivarius.

In embodiments, where acetate producing bacteria are included in the bacterial population, these may produce acetate via one or more of the following intermediate metabolites: phosphoenolpyruvate, pyruvate, formate, acetyl CoA.

In embodiments, where lactate producing bacteria are included in the bacterial population, these may produce lactate via one or more of the following intermediate metabolites: phosphoenolpyruvate, pyruvate.

In embodiments, where formate producing bacteria are included in the bacterial population, these may produce formate via one or more of the following intermediate metabolites: phosphoenolpyruvate, pyruvate.

In some embodiments, the bacterial population may comprise butyrate-producing bacteria from one or more of the families Lachnospiraceae and/or Ruminococcaceae. In some embodiments, the bacterial population may comprise butyrate-producing bacteria from one or more of the genera Butyricicoccus, Eubacterium, Anaerostipes, Coprococcus, Butyrivibrio, Roseburia, Faecalibacterium, Bariatricus, Megasphaera, and/or Clostridium. In preferred embodiments, the bacterial population may comprise butyrate-producing bacteria from one or more of the following species Butyricicoccus pullicaecorum, Butyrivibrio fibrisolvens, Eubacterium rectale, Eubacterium ramulus, Clostridium butyricum, Eubacterium limosum, Coprococcus catus, Coprococcus eutactus, Coprococcus comes, Dorea longicatena, Eubacterium cylindroides, Eubacterium hallii, Faecalibacterium prausnitzii, Anaerostipes hadrus, Anaerostipes rhamnosivorans, Anaerostipes caccae, Clostridium innocuum, Roseburia hominis, Roseburia faecis, Roseburia inulinivorans, Megasphaera elsdenii, Roseburia faecis, Roseburia intestinalis and/or Bariatricus massiliensis. Butyrate is a major short chain fatty acid that is present in the human intestinal environment, and the inventors have found that the presence of a butyrate producing species is important to accuratly simulate the intestinal environment. These preferred species of bacteria are known to produce butyrate and are therefore useful in simulating the intestinal environment.

In more preferred embodiments, the bacterial population may comprise butyrate-producing bacteria from one or more of the following species, Bacteroides massiliensis, Clostridium innocuum, Bariatricus massiliensis Eubacterium hallii, Dorea longicatena, Anaerostipes hadrus, Faecalibacterium prausnitzii, Roseburia faecis and/or Eubacterium rectale. As shown in the examples a bacterial population comprising Bariatricus massiliensis, Clostridium innocuum, Eubacterium hallii, Dorea longicatena, Roseburia faecis and Eubacterium rectale can produce butyrate in the simulated intestinal environment of the present invention.

In some embodiments, the bacterial population may comprise propionate-producing bacteria from one or more of the families Bacteroidaceae, Prevotellaceae, Rikenellaceae, Ruminococcaceae, Veillonellaceae and/or Verrucomicrobiaceae.

In some embodiments, the bacterial population may comprise propionate-producing bacteria from one or more of the genera Firmicutes, Verrucomicrobia, Bacteroidetes, Bacteroides, Blautia, Prevotella, and/or Eubacterium.

In some embodiments, the bacterial population may comprise propionate-producing bacteria from one or more of the following species: Akkermansia muciniphila, Bacteroides uniform is, Bacteroides vulgatus, Bacteroides dorei, Bacteroides thetaiotamicron, Prevotella copri, Dorea longicatena, Alistipes putredinis, Roseburia inulinivorans, Eubacterium hallii, Blautia obeum, Blautia sp., Coprococcus catus, Dialister invisus, Phascolarctobacterium succinatutens, Akkermansia muciniphila, Selenomonas ruminantium, Clostridium propionicum and/or Veillonella parvula.

In more preferred embodiments, the bacterial population may comprise propionate-producing bacteria from one or more of the following species: Dorea longicatena, Bacteroides thetaiotaomicron, Bacteroides uniform is, Bacteroides vulgatus, Bacteroides dorei, Bifidobacterium longum, Blautia sp, Eubacterium hallii, Prevotella copri, Prevotella sp. and/or Lactobacillus salivarius.

Prevotella copri, Blautia obeum, Bacteroides dorei, Bacteroides uniformis, Bacteroides thetaiotamicron and/or Bacteroides vulgatus are examples of organisms which may be employed in the simulated intestinal environment of the present invention.

In some embodiments, the bacterial population may comprise acetate-producing bacteria from the phylum Bacteroidetes. In certain embodiments, the bacterial population may comprise acetate-producing bacteria from one or more of the following genera Bacteroides, Bifidobacterium, Prevotella, Blautia, Selenomonas and/or Clostridium. In certain embodiments, the bacterial population may comprise acetate-producing bacteria from one or more of the following species: Bifidobacterium longum, Prevotella copri, Prevotella sp., Blautia coccoides, Bacteroides coccoides, Bacteroides dorei, Blautia producta, Bacteroides vulgatus, Bacteroides thetaiotamicron, Bacteroides uniformis, Bacteroides thetaiotaomicron, Selenomonas ruminantium, Lactobacillus salivarius and/or Bacteroides dorei.

In more preferred embodiments, the bacterial population may comprise acetate-producing bacteria from one or more of the following species: Bifidobacterium longum, Prevotella copri, Bacteroides dorei, Blautia producta, Bacteroides vulgatus, Bacteroides thetaiotamicron, Bacteroides uniformis, Bacteroides thetaiotaomicron and/or Lactobacillus salivarius.

Bacteroides thetaiotaomicron, Bacteroides uniformis, Bacteroides vulgatus, Bacteroides coccoides, Bacteroides dorei, Bifidobacterium longum, Lactobacillus salivarius and Prevotella copri and are examples of organisms which may be employed in the simulated intestinal environment of the present invention.

Additionally or alternatively, the bacterial population may comprise lactate-producing bacteria from one or more of the genera Bifidobacterium, Bacteroides, Anaerostipes, Coprococcus, Clostridium, Collinsella, Selenomonas, Roseburia and/or Lactobacillus.

In some embodiments, the bacterial population may comprise lactate-producing bacteria from one or more of the following species: Selenomonas ruminantium, Bifidobacterium adolescentis, Eubacterium rectale, Faecalibacterium prausnitzii, Anaerostipes caccae, Anaerostipes coli, Bifidobacterium longum, Anaerostipes hadrus, Dorea longicatena, Lactobacillus salivarus, Coprococcus catus, Bariatricus massiliensis and/or Clostridium indolis.

In more preferred embodiments, the bacterial population may comprise lactate-producing bacteria from one or more of the following species: Eubacterium rectale, Bifidobacterium longum, Lactobacillus salivarus and/or Bariatricus massiliensis.

Bifidobacterium longum and/or Lactobacillus salivarus are examples of organisms which may be employed in the simulated intestinal environment of the present invention.

In embodiments, the bacterial population may comprise formate-producing bacteria from the genera Escherischia, Ruminococcus and/or Bifidobacterium.

In some embodiments, the bacterial population may comprise formate-producing bacteria from one or more of the following species: Ruminococcus albus, Bifidobacterium longum and/or Escherichia coli. Escherichia coli and Bifidobacterium longum are examples of organisms which may be employed in the simulated intestinal environment of the present invention.

In addition to the short chain fatty acid-producing bacteria, organisms having additional functions may be included within the bacterial population. Thus, in embodiments of the invention, the bacterial population may comprise:
i) Sulphate releasing bacteria, such as sulphate-releasing bacteria from the genus Akkermansia. Akkermansia muciniphila is an example of an organism which may be employed in the simulated intestinal environment of the present invention.
ii) Mucin degrading bacteria, such as mucin degrading bacteria from the genus Akkermansia. Akkermansia muciniphila is an example of an organism which may be employed in the simulated intestinal environment of the present invention.
iii) Sulphate reducing bacteria, such as sulphate-reducing bacteria from the genus Desulfovibrio. Desulfovibrio piger is an example of an organism which may be employed in the simulated intestinal environment of the present invention.

iv) Methanogenic bacteria, such as methanogenic bacteria from the genus Methanobrevibacter. Methanobrevibacter smithii is an example of an organism which may be employed in the simulated intestinal environment of the present invention.
v) Ethanol producing bacteria, such as bacteria from the genus Escherichia. Escherichia coli is an example of an organism which may be employed in the simulated intestinal environment of the present invention.

In embodiments of the invention, the bacterial population present in the simulated intestinal environment of the present invention principally or exclusively comprises organisms which are commensal organisms isolated from the human intestine of a healthy donor. In such embodiments, at least 50%, at least 60%, at least 70%, at least 80%, at least 90%, at least 95%, at least 98%, at least 99% or at least 99.5% of the bacterial species present in the bacterial population are commensal organisms isolated from the human intestine of a healthy donor. The advantage of using commensal organisms isolated from the human intestine of a healthy donor for the bacterial population in the simulated intestinal environment is that these organisms can provide a more accurate model of the human gut microbiome. Methods for isolating commensal bacterial species are well known in the art.

Dramatic changes in microbiota composition have been documented in numerous diseases such as gastrointestinal disorders, for example inflammatory bowel disease (IBD) (Frank et al. (2007) PNAS 104(34):13780-5). In other embodiments of the invention, the bacterial population comprises organisms which are commensal organisms isolated from the human intestine of an unhealthy and/or diseased donor (such as a donor with irritable bowel syndrome). It can be advantageous to use bacterial populations isolated from an unhealthy and/or diseased donor, because this allows the simulated intestinal environment to emulate a diseased state. This allows for the accurate prediction of in vivo metabolic activity of an organism of interest in an intestinal environment that simulates the gut microbiome in an unhealthy human.

In certain embodiments of the invention, the bacterial organisms can be isolated from the human intestine of a single healthy donor of interest or multiple healthy donors of interest. In other embodiments, the bacterial organisms can be isolated from the human intestine of a single unhealthy and/or diseased donor of interest or multiple unhealthy and/or diseased donors of interest. In some embodiments the bacterial species can be isolated from at least 2 or more, 3 or more, 4 or more, 5 or more, 6 or more, 7 or more, 8 or more, 9 or more, 10 or more, 11 or more, 12 or more, 13 or more, 14 or more, 15 or more or 16 or more separate donors of interest.

The commensal organisms isolated from the human intestine of a healthy donor can be isolated as individual species of bacteria or as a combination of bacterial species. The commensal organisms isolated from the human intestine of a healthy donor can be isolated as individual strains of bacteria or as a combination of strains of bacteria. The individual species or strains of bacteria can be isolated depending on their metabolic function or growth profile using methods well known in the art, for example, using NMR, liquid chromatography, gas chromatography, liquid chromatography mass spectrometry or gas chromatography mass spectrometry.

Once the individual species or strains of bacteria or combination of bacterial species or strains have been isolated from the human intestine of a healthy or unhealthy donor of interest they can be used to inoculate an simulated intestinal environment or the bacteria can be stored using methods well-known in the art, for example a glycerol stock of the bacteria can be prepared and stored at −80° C. or the bacteria can be freeze dried. The simulated intestinal environment can be initiated with bacterial organisms isolated freshly from the human intestine of a healthy donor or the simulated intestinal environment can be initiated with bacterial organisms that have been freeze dried after isolation.

The simulated intestinal environment can be inoculated with separate individual species of bacteria or combinations of bacterial species. In other embodiments, the simulated intestinal environment can be inoculated with separate individual strains of bacteria or combinations of bacterial strains. The combinations of bacterial strains can include bacterial strains from the same species or can include bacteria strains from different species. A person skilled in the art can choose the specific species of bacteria that are used to inoculate the simulated intestinal environment. This allows for better control of the simulated intestinal environment.

The simulated intestinal environment of the invention can be used to simulate different compartments of the human gastrointestinal system such as, for example, the small intestine, the ascending colon, the transverse colon or the descending colon. The simulated intestinal environment of the invention can be used to simulate the microbiota found in each of these compartments. This ability to simulate the human gut microbiota in different gastrointestinal compartments is useful as it can provide valuable insights into the metabolic activity of cells of interest in different compartments of the human gut.

In certain embodiments of the invention, the bacterial population present in the simulated intestinal environment of the invention principally or exclusively comprises organisms which are not genetically modified organisms. In such embodiments, at least 50%, at least 60%, at least 70%, at least 80%, at least 90%, at least 95%, at least 98%, at least 99% or at least 99.5% of the bacteria present in the bacterial population are not genetically modified organisms.

In some embodiments of the invention, the bacterial population present in the simulated intestinal environment of the invention principally or exclusively comprises organisms which are not pathogenic. In such embodiments, at least 50%, at least 60%, at least 70%, at least 80%, at least 90%, at least 95%, at least 98%, at least 99% or at least 99.5% of the bacteria present in the bacterial population are not pathogenic.

One advantage of the simulated intestinal environment of the present invention is that it permits the ratio of bacteroides : firmicutes to be reliably controlled. Thus, in embodiments of the invention, the simulated intestinal environment comprises a bacterial population in which the ratio of bacteroidetes : firmicutes is 5 to 50:50 to 95, 10 to 45:55 to 90, 15 to 40:60 to 85, or 20 to 35:65 to 80. In preferred embodiments the ratio of bacteroidetes: firmicutes is 30-35:65-70, such as 30:65, 30:70, 35:65 or 35:70.

In some embodiments, the bacterial population present in the simulated intestinal environment contains both gram negative and gram positive bacteria. In certain embodiments the ratio of gram negative to gram positive bacteria is 20%:80%, 30%:70%, 40%:60%, 50%:50%, 60%:40%, 70%:30% or 80%:20%. In preferred embodiments the ration of gram negative to gram positive bacteria is 40%:60%, 50%:50% or 60%:40%. In alternative embodiments the bacterial population contains only gram negative bacteria. In other embodiments the bacterial population contains only gram positive bacteria.

In terms of the composition of the liquid medium, this may comprise materials conventionally used to prepare liquid growth media. The exact composition of the medium is not critical. Rather, any medium which permits stabilisation of the bacterial population can be used.

Examples of liquid medium that can be used include: YFCA, Postgate's medium, Cooked meat broth (e.g. Robertson's Cooked Meat Medium), Peptone-yeast extract glucose broth, Thioglycollate broth, Lysogeny Broth (LB) medium, Minimal salts (M9) medium, Terrific Broth, SOB medium, SOC medium, 2X YT medium, NZCYM Broth, NZYM Broth, NZM Broth, Tryptic soy Broth and SIEM medium. In preferred embodiments the medium is YFCA, Postgate's medium, Cooked meat broth (e.g. Robertson's Cooked Meat Medium), Peptone-yeast extract glucose broth, Thioglycollate broth and SIEM medium. Most preferably the medium is YCFA.

In certain embodiments the YFCA medium contains per litre Casein hydrolysate 10.0 g, Yeast Extract 2.5 g, Sodium hydrogen carbonate 4.0 g, Glucose 2.0 g, Cellobiose 2.0 g, Soluble starch 2.0 g, Di-potassium hydrogen phosphate 0.45 g, Potassium di-hydrogen phosphate 0.45 g, Resazurin 0.001 g, L-Cysteine HCl 1.0 g, Ammonium sulphate 0.9 g, Sodium chloride 0.9 g, Magnesium sulphate 0.09 g, Calcium chloride 0.09 g, Haemin 0.01 g, SCFA 3.1 ml (Acetic acid 2.026 ml/L, Propionic acid 0.715 ml/L, n-Valeric acid 0.119 ml/L, Iso-Valeric acid 0.119 ml/L, Iso-Butyric acid 0.119 ml/L), vitamin mix 1:1 ml (Biotin 1 mg/100 ml, Cyanocobalamine 1 mg/100 ml, p-Aminobenzoic acid 3 mg/100 ml, Pyridoxine 15 mg/100 ml ), vitamin mix 2:1 ml (Thiamine 5 mg/100 ml, Riboflavin 5 mg/100 ml ), vitamin mix 3:1 ml (Folic acid 5 mg/100 ml).

Additional components may be included in the liquid medium. The purpose of these may be to mimic gastrointestinal conditions. In some embodiments, mucin may be included for example at a level of about 0.001%, about 0.002%, or about 0.005% to about 0.05%, about 0.1%, about 0.2%, about 0.5% or about 1% by weight of the liquid medium.

To maintain optimal conditions for survival and operation of the bacterial population, the simulated intestinal environment of the present invention preferably has a pH of 4 or higher, 4.5 or higher, 5 or higher, 5.5 or higher or 6 or higher. Additionally or alternatively, the simulated intestinal environment of the present invention preferably has a pH of 8 or lower, 7.5 or lower, 7.0 or lower or 6.5 or lower.

Additionally or alternatively, the simulated intestinal environment of the present invention is preferably stored under anaerobic conditions, e.g. in an atmosphere comprising less than about 500 ppm, less than about 200 ppm, less than 100 ppm, less than 50 ppm or less than about 20 ppm of oxygen.

The nutritional energy source comprised within the simulated intestinal environment of the present invention may comprise any compounds which can be metabolised by the bacterial population (either directly or indirectly) to produce at least three of butyrate, propionate, acetate, lactate and formate.

For example, the nutritional energy source may comprise saccharides including monosaccharides, disaccharides and/or oligosaccharides. Preferred examples of such saccharides that may be comprised in the nutritional energy source in the present invention include fucose, rhamnose, hexose, pentose. Specific examples of pentose saccharides that may be employed in the present invention are arabinose, lyxose, ribose, xylose, ribulose, xylulose, and deoxyribose. Specific examples of hexose saccharides that may be employed in the present invention are allose, altrose, glucose, mannose, gulose, idose, galactose, talose, psicose, fructose, sorbose, and tagatose. In embodiments of the invention, the nutritional energy source may be present in amounts of less than about 2%, less than about 1%, less than about 0.5%, less than about 0.2% or less than 0.1% by weight of the liquid medium. Additionally or alternatively, the nutritional energy source may be present in amounts of more than about 0.001%, about 0.002%, about 0.005%, about 0.01%, 0.02% or 0.05% by weight of the liquid medium.

As explained above, the simulated intestinal environment of the present invention advantageously permits the metabolic activity of a cell of interest to be assessed in a controlled and straightforward manner.

The examples demonstrate that when Megasphaera massiliensis was added to the simulated intestinal environment of the present invention the metabolites and specific HDAC inhibitory effects of this strain were transferred to the bacterial core community. This demonstrates that the metabolic activity of a cell of interest can successfully be transferred into the simulated intestinal environment of the present invention, which allows the metabolic properties of the cell of interest to be analysed in a controlled environment.

According to another aspect of the present invention, a process is provided for making a stabilised simulated intestinal environment according to the invention and as described above. Said process comprises inoculating a liquid medium with a bacterial population consisting essentially of 5 to 200 species of bacteria wherein said bacterial population is capable of producing at least three of butyrate, propionate, succinate, ethanol, acetate, lactate and formate from said nutritional energy source. In preferred embodiments, the process for making a stabilised simulated intestinal environment comprising inoculating a liquid medium with a bacterial population consisting essentially of 5 to 20 species of bacteria. In preferred embodiments the strains of bacteria are chosen from the following species: Alistipes putredinis, Akkermansia muciniphila, Anaerostipes caccae, Anaerostipes coli, Anaerostipes hadrus, Anaerostipes rhamnosivorans, Bacteroides coccoides, Bacteroides dorei, Bacteroides massiliensis, Bacteroides thetaiotamicron, Bacteroides uniformis, Bacteroides vulgatus, Bariatricus massiliensis, Bifidobacterium adolescentis, Bifidobacterium longum, Blautia producta, Blautia coccoides, Blautia obeum, Butyricicoccus pullicaecorum, Butyrivibrio fibrisolvens, Clostridium butyricum, Clostridium indolis, Clostridium innocuum, Clostridium propionicum Coprococcus catus, Coprococcus comes, Coprococcus eutactus, Dialister invisus, Desulfovibrio piger, Dorea longicatena, Escherichia coli, Eubacterium cylindroides, Eubacterium hallii, Eubacterium limosum, Eubacterium ramulus, Eubacterium rectale, Faecalibacterium prausnitzii, Lactobacillus salivarius, Megasphaera elsdenii, Methanobrevibacter smithii, Phascolarctobacterium succinatutens, Prevotella copri, Ruminococcus albus, Roseburia faecis, Roseburia hominis, Roseburia intestinalis, Roseburia inulinivorans, Selenomonas ruminantium and/or Veillonella parvula.

It is understood that this process will generally be an in vitro process.

As demonstrated in the examples, the inventors have shown that the order in which the bacterial species are added to the simulated intestinal environment can be important. The order in which the bacterial species are inoculated is selected based on the sensitivity and growth profile of the bacterial species. For example, bacteria that grow very well and very quickly in any condition (such as E. coli) are inoculated later to ensure that the more sensitive or strictly anaerobic bacteria (such as Eubacterium rectale, Roseburia faecis, Dorea longicatena, Eubacterium hallii and/or Bariatricus massiliensis) are established in the simulated intestinal environment before more highly competitive bacteria species are added. This process is advantageous as it prevents the simulated intestinal environment from being dominated by a single or small number of bacterial species and allows the simplified intestinal environment of the invention to more accurately simulate the human microbiome, which contains a wide range of bacterial species.

Calculating the specific growth rate of a bacterial species is well-known in the art. For example a spectrophotometer can be used to measure the turbidity or optical density of the bacterial suspension, where an increase in turbidity indicates an increase in bacterial biomass.

Thus, in certain embodiments the simulated intestinal environment is sequentially inoculated with the bacterial species of the invention as described above. In particular, a process for preparing a simulated intestinal environment of the invention may comprise a step of inoculation with 5 to 200 species of bacteria wherein the bacterial species are added sequentially based on their growth characteristics wherein the bacterial species which show slower growth characteristics are preferentially added before the bacterial species with faster growth characteristics. A skilled person will understand that the growth characteristics of the individual species are assessed under the growth conditions which are found in the stabilised simulated intestinal environment. This can be determined easily by assessing the different bacteria's growth characteristics in the stabilised simulated intestinal environment in a control experiment. “Preferentially” in this context also does not mean that the bacteria have to be added strictly in order of their growth characteristics. Rather it means that the majority of bacteria with slower growth characteristics (e.g. at least 90%, at least 95%, or at least 99%) are added before bacteria with faster growth characteristics.

In certain embodiments, the simulated intestinal environment is inoculated with individual species of bacteria. In other embodiments, the simulated intestinal environment is inoculated with at least 2 or more, 3 or more, 4 or more, 5 or more, 6 or more, 7 or more, 8 or more, 9 or more, 10 or more, 11 or more, 12 or more, 13 or more, 14 or more or 15 or more groups of bacteria. In certain embodiments, the groups of bacterial have similar growth profiles, for example species of bacteria in a group will all be strictly anaerobic. In preferred embodiments, the simulated intestinal environment is inoculated with at least 7 or more groups of bacteria.

Thus, according to a further aspect of the present invention, there is provided a process for assessing the metabolic activity of a cell of interest comprising the steps of:
providing a stabilised simulated intestinal environment comprising a liquid medium as described herein, a bacterial population as described herein and a nutritional energy source also as described herein,
adding the cell of interest to the simulated intestinal environment, and
assessing the composition of the simulated intestinal environment following addition of the cell of interest.

In certain embodiments the process can comprise a further step of maintaining the simulated intestinal environment under stabilisation conditions until stabilisation of the intestinal environment is attained.

In other aspects of the invention the organism of cell of interest can be added to the simulated intestinal environment before stabilisation of the intestinal environment is attained. As discussed above the bacterial species in the bacterial population can be added sequentially in multiple groups. The cell of interest can be added to any one of these groups. In preferred embodiments, as shown in the examples the cell of interest is added as the last group to the simulated intestinal environment. If the bacterial species is strictly anaerobic or does not grow very well it may be advantageous to add the cell of interest earlier to the simulated intestinal environment, for example in group 2, 3 or 4.

The composition of the simulated intestinal environment can be assessed to observe changes that occur after the addition of the cell of interest. These changes can include increases or decreases in the levels of metabolites produced by the simulated intestinal environment, such as butyrate, propionate, succinate, ethanol, acetate, lactate and formate. Methods for measuring metabolites are well known in the art, for example, using NMR, liquid chromatography, gas chromatography, liquid chromatography mass spectrometry or gas chromatography mass spectrometry. As shown in the examples the addition on the cell of interest can lead to an additional metabolite being produced by the simulated intestinal environment. The process of the present invention allows the metabolic activity of the organism of interest to be elucidated in a simplified simulated intestinal environment.

Thus in certain embodiments, the invention provides a process for assessing the metabolic activity of a cell of interest comprising the steps of:
providing a simulated intestinal environment comprising a liquid medium as described herein, a bacterial population as described herein and a nutritional energy source also as described herein and a cell of interest, and
assessing the composition of the simulated intestinal environment following addition of the cell of interest.

In certain embodiments the process can comprise a further step of maintaining the simulated intestinal environment under stabilisation conditions until stabilisation of the intestinal environment is attained.

The process of the present invention may be used to investigate the metabolic activity and potentially elucidate the mechanism of action of any type of cell including prokaryotic or eukaryotic cells. In embodiments of the invention, the cell of interest may be a bacterial cell, a fungal cell, an archaeal cell or a virus. The cell of interest may be pathogenic. Alternatively, the cell of interest may be beneficial to health; in such embodiments, the present invention advantageously finds utility as part of the drug discovery process.

The process of the present invention allows the metabolic activity of the organism of interest to be elucidated in a simplified simulated intestinal environment. An isolated organism of interest will have a certain metabolic profile when studied individually. However, the human gut microbiome is made up of thousands of different bacterial species that interact with each other, for example competing for nutrients or working symbiotically. The inventors have identified, however, that the metabolites butyrate, propionate, succinate, ethanol, acetate, lactate and/or formate are of particular importance when studying the microbiome in a simulated intestinal environment.

The process of the present invention allows the metabolic activity of an organism to be elucidated in a controlled environment that simulates the intestinal environment. The process of the present invention allows a reliable prediction of the in vivo metabolic properties of an organism of interest in a gut microbial community.

In embodiments of the invention, for example in situations where the user wishes to investigate the metabolic activity of a cell with a particular substrate or a second cell of interest, the process of the invention may comprise the step of adding a substrate and/or a second cell of interest to the simulated intestinal environment. The second cell of interest may be added to the simulated intestinal environment after attainment of stabilisation of the simulated intestinal environment.

In embodiments of the invention, the process of the invention can be used to screen organisms of interest for specific metabolite activity in a simulated intestinal environment. Screening for specific metabolite activity in this way allows a reliable prediction of the in vivo metabolic properties of an organism of interest in a gut microbial community. In other embodiments, the co-incubation of the simulated intestinal environment with a metabolite of interest can be performed, for example with secondary plant metabolites. The process of the invention allows a reliable prediction of the in vivo effect of a metabolite of interest can have on the human gut microbiome.

The substrate may be added to the simulated intestinal environment before or after attainment of stabilisation of the simulated intestinal environment. Where the substrate is a saccharide, it is preferably not a saccharide which is metabolised by the bacterial population and/or is not a saccharide comprised in the nutritional energy source.

As will be appreciated by those skilled in the art, it is desirable for the simulated intestinal environment to attain stabilisation as quickly as possible so that set-up time is minimised and analysis of the metabolic properties of the cell of interest can be commenced as promptly as possible. As is demonstrated in the examples which follow, the stabilisation of the simulated intestinal environment of the present invention can be achieved significantly more rapidly than with prior art arrangements. Thus, in embodiments of the present invention, stabilisation is achieved in 2 weeks or less, in 10 days or less, in 1 week or less or in 5 days or less, or in 4 days or less, or in 3 days or less, or in 2 days or less, or in 1 day or less.

Any stabilisation conditions may be employed provided that stabilisation of the simulated intestinal environment is attained. In embodiments of the invention, the stabilisation conditions may include one or more of the following:
temperature control—the temperature of the simulated intestinal environment may be maintained at a temperature of from about 20° C., about 25° C. or about 30° C. to about 40° C., about 45° C. or about 50° C.
pH control—the pH of the simulated intestinal environment may be maintained at a pH of 4 or higher, 4.5 or higher, 5 or higher, 5.5 or higher or 6 or higher to about a pH of 8 or lower, 7.5 or lower, 7.0 or lower or 6.5 or lower.
Atmospheric control—the environment in which the simulated intestinal environment is maintained under stabilisation conditions may be anaerobic, e.g. the simulated intestinal environment may be maintained in an atmosphere comprising less than about 500 ppm, less than about 200 ppm, less than 100 ppm, less than 50 ppm or less than about 20 ppm of oxygen.

As explained above, the simulated intestinal environment is maintained at stabilisation conditions until stabilisation of the simulated intestinal environment is attained. For the purposes of the present invention, stabilisation is deemed to have been attained once at least three of butyrate, propionate, acetate, lactate and formate are present in the simulated intestinal environment and the concentrations of each of those compounds do not change by 20% over a 24 hour period.

In some embodiments, the simulated intestinal environment comprises at least three of butyrate, propionate, acetate, lactate and formate, which can be produced by the bacterial population and/or added directly to the liquid medium. Prior to stabilisation the total levels of metabolites, such as butyrate, propionate, acetate, lactate and formate, produced by the bacterial population in the simulated intestinal environment vary. When stabilisation is attained at least three of butyrate, propionate, acetate, lactate and formate are produced by the bacteria in the composition and the concentrations of each of those compounds do not change by 20% over a 24 hour period, a 18 hours period, a 12 hour period or a 6 hour period.

In embodiments in which the simulated intestinal environment, following the attainment of stabilisation, comprises butyrate, butyrate may be present at a concentration of at least about 0.5 mM, about 1.0 mM, about 1.5 mM, about 2.0 mM, about 3.0 mM, about 4.0 mM or about 5.0 mM. Additionally or alternatively, butyrate may be present at a concentration of about 10.0 mM or lower, about 8.0 mM or lower, about 6.0 mM or lower, or about 5.0 mM or lower. In a preferred embodiment in which simulated intestinal environment, following the attainment of stabilisation, comprises butyrate, butyrate is present at a concentration of about 1.0 mM to about 5.0 mM.

In embodiments in which the simulated intestinal environment, following the attainment of stabilisation, comprises propionate, propionate is present at a concentration of at least about 0.5 mM, about 1.0 mM, about 1.5 mM, about 2.0 mM, about 3.0 mM, about 4.0 mM or about 5.0 mM. Additionally or alternatively, propionate may be present at a concentration of about 10.0 mM or lower, about 8.0 mM or lower, about 6.0 mM or lower, or about 5.0 mM or lower. In a preferred embodiment in which the simulated intestinal environment, following the attainment of stabilisation, comprises propionate, propionate is present at a concentration of about 2.0 mM to about 7.0 mM.

In embodiments in which the simulated intestinal environment, following the attainment of stabilisation, comprises acetate, acetate is present at a concentration of at least about 0.1 mM, about 0.2 mM, about 0.5 mM, about 1.0 mM, about 1.5 mM, or about 2.0 mM. Additionally or alternatively, acetate may be present at a concentration of about 5.0 mM or lower, about 4.0 mM or lower, about 3.0 mM or lower, or about 2.0 mM or lower. In a preferred embodiment in which the simulated intestinal environment, following the attainment of stabilisation, comprises acetate, acetate is present at a concentration of about 0.2 to about 2.0 mM.

In embodiments in which the simulated intestinal environment, following the attainment of stabilisation, comprises lactate, lactate is present at a concentration of at least about 0.1 mM, about 0.2 mM, about 0.5 mM, about 1.0 mM, about 1.5 mM, or about 2.0 mM. Additionally or alternatively, lactate may be present at a concentration of about 5.0 mM or lower, about 4.0 mM or lower, about 3.0 mM or lower, or about 2.0 mM or lower. In a preferred embodiment in which the simulated intestinal environment, following the attainment of stabilisation, comprises lactate, lactate is present at a concentration of about 0.2 to about 2.0 mM.

In embodiments in which the simulated intestinal environment, following the attainment of stabilisation, comprises formate, formate is present at a concentration of at least about 0.01 mM, about 0.02 mM, about 0.05 mM, about 0.1 M, about 0.2 mM, or about 0.5 mM.

Additionally or alternatively, formate may be present at a concentration of about 2.0 mM or lower, about 1.5 mM or lower, or about 1.0 mM or lower. In a preferred embodiment in which the simulated intestinal environment, following the attainment of stabilisation, comprises formate, formate is present at a concentration of about 0.01 to about 0.5 mM.

The assessment of the composition of the simulated intestinal environment following addition of the cell of interest can be carried out using techniques known to those skilled in the art. In embodiments of the invention, metabolomic analysis may be carried out, for example to determine the concentrations of short chain fatty acids and medium chain fatty acids, including formate, acetate, propionate, butyrate, isobutyrate, valerate, isovalerate, hexanoate, octanoate, decanoate, dodecanoate. Metabolomic analysis may be carried out using any technique known to those skilled in the art, for example HPLC.

Additionally or alternatively, the assessment of the composition of the simulated intestinal environment may comprise quantifying the numbers of cell of interest, so that the user can assess growth rates and optimal growth conditions of that cell.

Changes to the bacterial population may also be assessed as part of the assessment of the simulated intestinal environment. For example, the proportions of the different members of the population can be assessed to predict the effect of exposing commensal bacteria to the cell of interest. Such an assessment can be made, for example, using 16 S metagenomic sequencing, a technique with which those skilled in the art will be familiar. The skilled person will also be familiar with apparatus which can be used to carry out such sequencing, for example Illumina MiSeq.

The assessment of the composition can be carried out at any time following the attainment of stabilisation of the simulated intestinal environment. However, those skilled in the art will recognise that it will be preferable to defer making the assessment until a timepoint at which the cell of interest has had sufficient time to influence the composition. For example, the assessment of the composition may be made 1 hour, 2 hours, 3 hours, 4 hours, 5 hours, 6 hours, 8 hours, 10 hours, 12 hours, 18 hours or 24 hours following the addition of the cell of interest to the simulated intestinal environment.

Thereafter, subsequent assessments may be made, optionally at periodic (e.g. 1 hour, 2 hour, 3 hour, 4 hour, 5 hour, 6 hour, 8 hour, 10 hour, 12 hour, 18 hour or 24 hour) intervals.

The assessment/s of the simulated intestinal environment may be made directly in the simulated intestinal environment itself (e.g. by analysing the simulated intestinal environment while in the fermenter), or the method of the invention may comprise the step of extracting a sample of the simulated intestinal environment and conducting the assessment on that sample.

In embodiments of the invention, the process further comprises the step of a control study in which a second simulated intestinal environment corresponding to the simulated intestinal environment is provided and maintained under stabilisation conditions corresponding to those used to attain stabilisation of the simulated intestinal environment, such that stabilisation of the second simulated intestinal environment is attained, wherein the composition of the second simulated intestinal environment is assessed in the same way as the simulated intestinal environment, wherein the cell of interest is not added to the second simulated intestinal environment, and wherein the results of the assessment of simulated intestinal environment are compared to the results of the assessment of the second simulated intestinal environment.

In such embodiments, the simulated intestinal environment and second simulated intestinal environment may be taken from a common stock of simulated intestinal environment. Alternatively, the simulated intestinal environment and second simulated intestinal environment may be separately prepared.

The process of the invention may be carried out in any suitable apparatus which permits the maintenance of a bacterial population at controlled conditions. In embodiments of the invention, the process is carried out in a fermenter (e.g. a stainless steel, plastic or glass fermenter) comprising heating means (e.g. a magnetic heater), a pH sensor, and/or a temperature sensor. The fermenter may be provided with one or more inlets permitting the addition of one or more of liquid medium, bacterial population, the cell of interest, substrate, additive/s, inert gas such as nitrogen and/or pH adjusting agents including acids and bases. Additionally or alternatively, the fermenter may be provided with one or more outlets for extracting samples of the simulated intestinal environment for testing, liquid effluent and/or waste gas.

In embodiments of the invention, the process is advantageously operated on a continuous basis. For example, liquid medium may be fed continuously or intermittently into the apparatus in which the bacterial population is maintained at controlled conditions. The rate at which the liquid medium is fed into the apparatus in which the bacterial population is maintained at controlled conditions may be about 0.1 ml/minute, 0.2 ml/minute, 0.3 ml/minute, about 0.4 ml/minute or about 0.5 ml/minute to about 1 ml/minute, 2 ml/minute, 3 ml/minute, 4 ml/minute or about 5 ml/minute.

Additionally or alternatively, samples may be extracted continuously or intermittently from the apparatus in which the bacterial population is maintained at controlled conditions. The rate at which the samples are extracted from the apparatus in which the bacterial population is maintained at controlled conditions may be about 0.1 ml/minute, 0.2 ml/minute, 0.3 ml/minute, about 0.4 ml/minute or about 0.5 ml/minute to about 1 ml/minute, 2 ml/minute, 3 ml/minute, 4 ml/minute or about 5 ml/minute.

In a further aspect of the invention, there is provided a kit comprising a bacterial population as described herein and instructions to prepare a simulated intestinal model as discussed herein. In certain embodiments, the bacterial population may be provided in the form of a lyophilised composition. Additionally or alternatively, the kit may comprise a nutritional energy source as described herein and/or a liquid medium as described herein.

### EXAMPLES

### Example 1—Rational Design of Bacterial Population

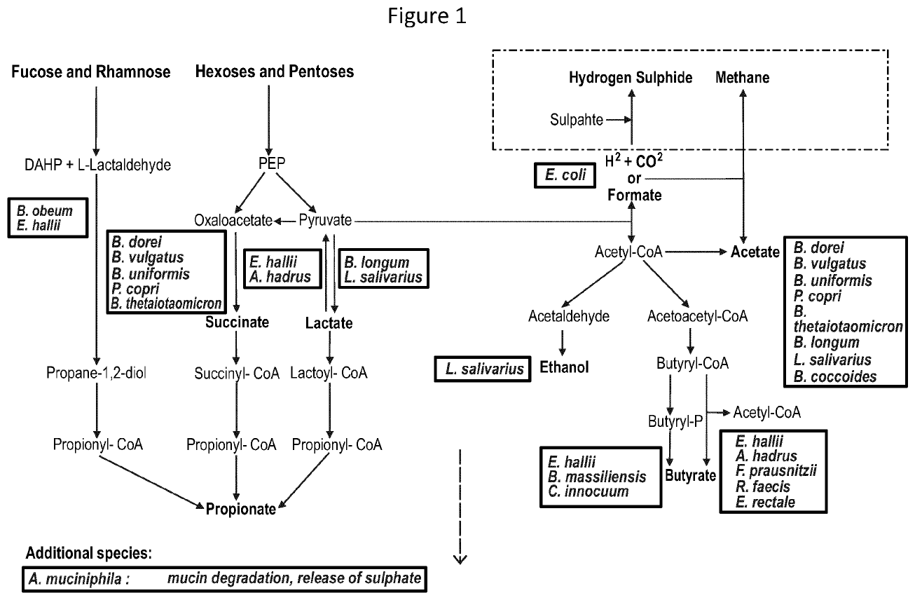

A bacterial population was designed with the aim of being simplified (as compared to bacterial populations employed in prior art simulated gastrointestinal environments) and enabling rapid attainment of stabilisation. As is demonstrated in FIG. 1, the bacterial population was designed to encompass all major metabolic pathways for the short chain fatty acids identified by the inventors as being of importance to assessing the metabolic activity of cells of interest. Further, the bacteria making up the population were selected to avoid overgrowth of specific organisms and out-competition of faster growing strains over slower growing strains.

### Example 2—Stabilisation of the Simulated Intestinal Environment

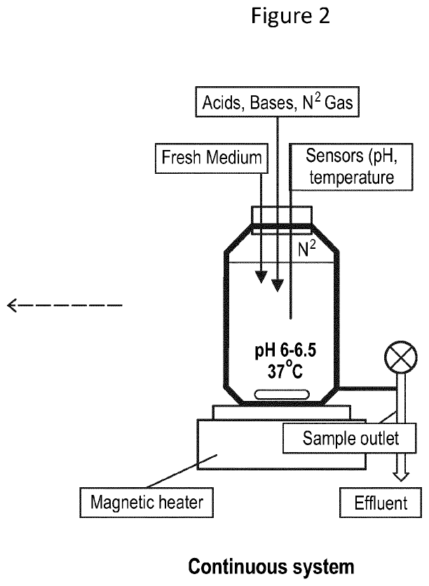

The apparatus shown schematically in FIG. 2 was set up. The illustrated apparatus comprises a glass fermenter (1) into which the simulated intestinal environment (3) has been provided. The simulated intestinal environment (3) comprises the bacterial population designed in Example 1, liquid medium and a nutritional energy source comprising fucose, rhamnose, hexoses and pentoses.

The fermenter (1) is sealed and operated under anaerobic conditions (nitrogen blanketing). The apparatus is advantageously figured to operate continuously and thus the fermenter (1) is provided with medium inlet (5) which permits the simulated intestinal environment to be maintained at constant volume and also control inlet (7) which permits the addition of acid and base to control pH as well as nitrogen to maintain the anaerobic environment. Sensor inlet (9) permits the insertion of pH and temperature sensors into the interior of the fermenter (1). The fermenter (1) is also provided with a magnetic heater (11).

Outlet (13) permits the extraction of samples of simulated intestinal environment for assessment as well as the removal of effluent.

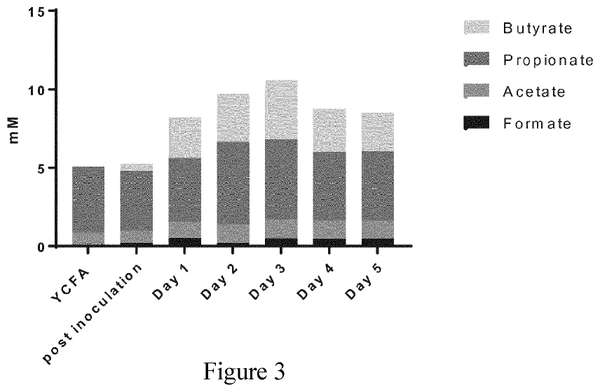

Following the addition of liquid medium, bacterial population and nutritional energy source into the fermenter (1), the simulated intestinal environment (3) was maintained at stabilisation conditions (pH 6 to 6.5, temperature of 37° C.) for five days. The levels of butyrate, propionate, acetate and formate were assessed upon the addition of the bacterial population to the simulated intestinal environment and then at 24 hour intervals thereafter and the results of these assessments are shown in FIG. 3. As can be seen, stabilisation of the simulated intestinal environment was attained after 4 to 5 days, which is considerably more rapid than with prior art arrangements.

### Example 3—Assessment of Efficacy of MRX0029 in a Simplified Model of the Human Gut Microbiota (SimMi)

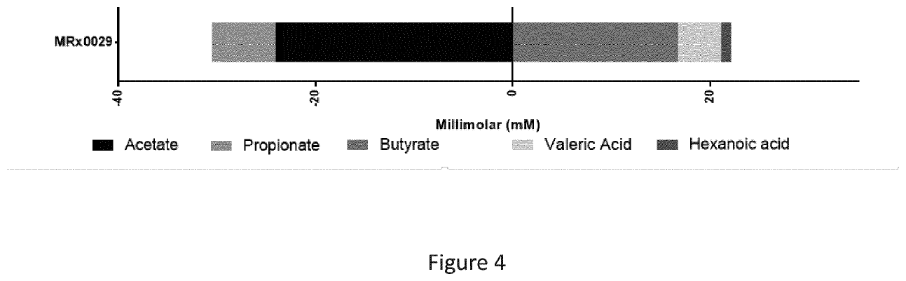

The Megasphaera massiliensis strain MRx0029 is a butyrate producing bacterial strain that can also produce valeric acid and the medium chain fatty acid (MCFA) hexanoic acid (see FIG. 4).

The ability of MRx0029 to maintain its short chain fatty acids SCFA and MCFA production and HDAC activity in an established bacterial community was tested in a simulated intestinal environment of the present invention (referred to herein as SimMi). This system was used to predict the in vivo metabolic properties of MRx0029.

### Methods

A stable community of 16 bacterial strains previously isolated from human faecal samples of healthy donors was developed in continuous culture to mimic core metabolic functions of the human gut microbiota as described in Examples 1 and 2.

The bacterial strains in the SimMi environment were Bacteroides dorei, Bacteroides uniformis, Bacteroides thetaiotaomicron, Bacteroides vulgatus, Bifidobacterium longum, Blautia producta, Blautia sp., Bariatricus massiliensis, Clostridium innocuum, Dorea longicatena, Eubacterium hallii, Escherichia coli, Eubacterium rectale, Lactobacillus salivarus, Prevotella sp. and Roseburia faecis.

The bacterial species in the SimMi environment cover a wide range of metabolic pathways, mainly focussed on SCFA production, but also considers cross-feeding, bacterial abundance and diversity.

The bacterial community was inoculated in seven groups.

| Group | | |
|---|---|---|
| number | Species in group | Inoculation time |
| 1 | Eubacterium rectale, Roseburia | — |
| | faecis, Dorea | |
| | longicatena, Eubacterium | |
| | hallii, Bariatricus | |
| | massiliensis | |
| 2 | Blautia producta, Blautia sp., | Group 2 is inoculated |
| | Bacteroides | 30 minutes |
| | uniformis, Bacteroides | after group 1 |
| | thetaiotaomicron, | |
| | Prevotella sp. | |
| 3 | Bacteroides dorei, | Group 3 is inoculated |
| | Bacteroides vulgatus | 20 minutes |
| | | after group 2 |
| 4 | Clostridium innocuum | Group 4 is inoculated |
| | | 10 minutes |
| | | after group 3 |
| 5 | Bifidobacterium longum | Group 5 is inoculated |
| | | 10 minutes |
| | | after group 4 |
| 6 | Lactobacillus salivarus | Group 6 is inoculated |
| | | 10 minutes |
| | | after group 5 |
| 7 | Escherichia coli | Group 7 is inoculated |
| | | 20 minutes |
| | | after group 6 |

The simulated intestinal environment was inoculated and allowed to establish for 1 hour prior to inoculation with Megasphaera massiliensis strain MRx0029. After the final inoculation the simulated bacterial environment is left for 2 hours and then the reactor vessel is then pumped with clean media at a flow rate of 0.5 mL/min.

SimMi cultures with and without MRX0029 were grown for 13 days.

### SCFA and MCFA Quantification of Bacterial Supernatants

The metabolism of the two consortia were analysed over the 13 days. Short chain fatty acids (SCFAs) and medium chain fatty acids (MCFAs) from bacterial supernatants were analysed and quantified by MS Omics APS, Denmark. Samples were acidified using hydrochloride acid, and deuterium labelled internal standards were added. All samples were analyzed in a randomized order. Analysis was performed using a high polarity column (Zebron™ ZB-FFAP, GC Cap. Column 30 m×0.25 mm×0.25 μm) installed in a gas chromatograph (7890B, Agilent) coupled with a quadropole detector (59977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described in Johnsen, L. G et al. (2017). J Chromatogr A, 1503: p. 57-64.

### HDAC Activity Assay

The HDAC activity of the samples on day 12 from the two SimMi cultures (with and without MRX0029) culture two consortia were analysed.

Cell free supernatants (CFS) from the SimMi cultures with and without MRX0029 were isolated for HDAC activity analyses. Aliquots of the two SimMi cultures (with and without MRX0029) were centrifuged at 5000 x g for 5 minutes and the cell-free supernatant (CFS) was filtered using a 0.2 μM filter (Millipore, UK), after which 1 mL aliquots of the CFS were stored at −80° C. until use.

Cell culture: HT-29 human colorectal adenocarcinoma cells were obtained from the European Collection of Cell Cultures (ECACC) (passage 162-173). Cells were grown in Dulbecco's minimum essential media (DMEM) media containing 10% FBS, 4 mM L-glutamine, 1% non-essential amino acids and antimycotic and antibiotic (Sigma, UK). Three days post-confluence, cells were washed twice with Hank's Balanced Salt Solution (HBSS) and stepped down in 1 mL of DMEM with 4 mM L-glutamine, 1% non-essential amino acids, 5 μgml-1 apo-transferrin and 0.2 μgml-1 sodium selenite (Sigma Aldrich, UK). Cells were stepped down 24 h prior to commencement of the experiment.

### HDAC Assay

HT-29 cells were incubated in a CO₂ incubator for 48 h with 100 μL of CFS from SimMi cultures with and without MRX0029.

### Nuclear Protein Extraction

After treatment the cells were washed twice with PBS and then harvested by scraping the cells from the wells. Cells were centrifuged at 450 x g for 5 min. Nuclear extractions were then conducted according to manufacturer's instructions using the NXTRACT NuCLEAR kit (Sigma Aldrich, UK). Once extracted, the nuclear proteins were snap-frozen and stored at −80° C. for HDAC activity analysis.

### Total HDAC Activity Analysis

HDAC activity was analysed using the histone deacetylase assay kit (Sigma Aldrich, UK). The assay was conducted according to manufacturer's instructions using 15 μL of extracted HT-29 nuclear protein.

Additionally, HT-29 nuclear protein of untreated cells was extracted and normalised to the protein concentration of a HeLa cell lysate provided with the NXTRACT NuCLEAR kit (Sigma Aldrich, UK). Protein concentrations were determined using the Pierce Bicinchoninic Protein Assay (BCA) kit A (Thermo Fisher, UK). 15 uL of this extract was used for HDAC activity analysis after incubation of 10% CFS, to confirm HDAC inhibition in whole cells.

### Results

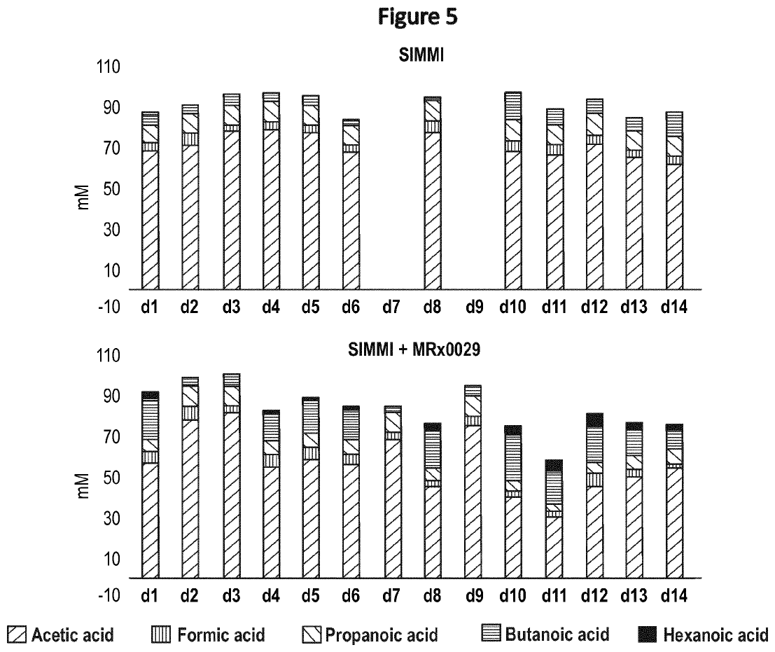

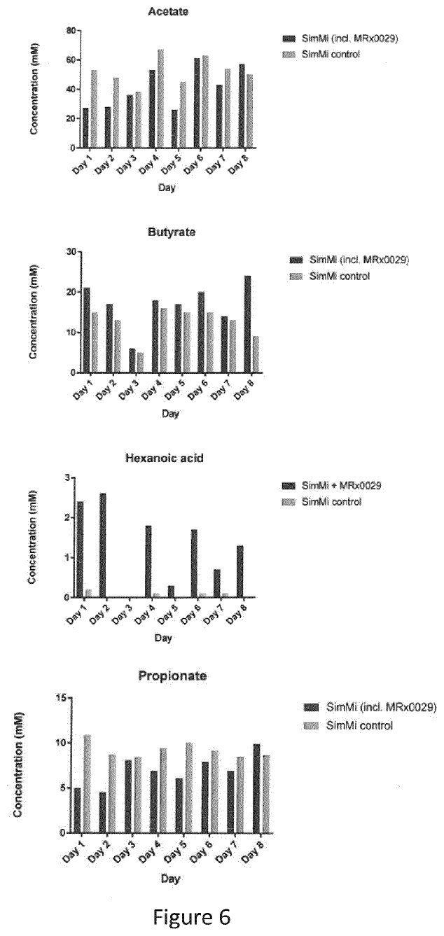

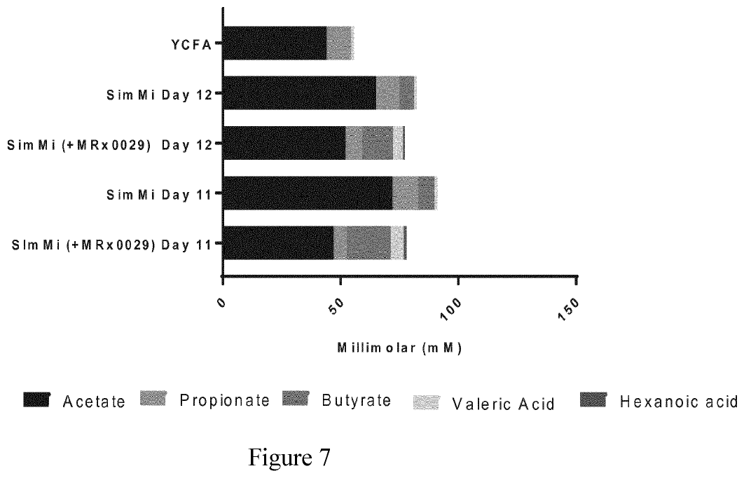

The addition of MRx0029 to the SimMi culture introduces additional pathways into the culture. Valeric acid and hexanoic acid were not produced in the original core consortium of SimMi, but were produced in the SimMi with added MRx0029 over the entire period of the run (13 days) (FIGS. 5-7).

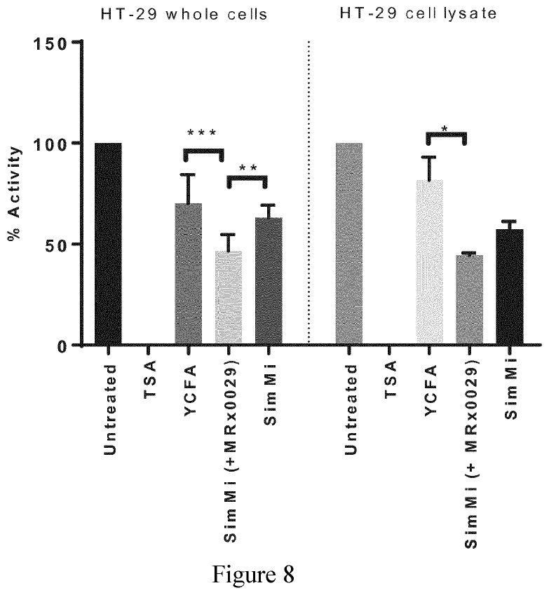

Aliquots from 12 of the SimMi and SimMi+MRX0029 cultures were tested for total HDAC inhibition with YCFA acting as control. The results demonstrate that the SimMi+MRX0029 culture have a more potent total HDAC inhibition than the standard consortium on whole HT-29 cells (p<0.001) and on HT-29 cell lysate (p<0.05) (FIG. 8). This indicates the physiologically relevant potential of MRx0029, as a butyrate and valeric acid producing bacteria and to stimulate HDAC inhibition within an established bacterial community are maintained in the SimMi culture.

Adding M. massiliensis MRx0029 to an in vitro simplified human gut microbiota consortium showed that metabolites and specific HDAC inhibitory effects of this strain were transferred to the bacterial core community

These results demonstrate the impact of a community on the efficacy of a cell of interest and vice versa. This permits an assessment to be made of what impact on the pharmacological characteristics a cell of interest will have in vivo, without having to conduct an in vivo study.

1-51. (canceled)

52. A method for an in vitro process for making a stabilized simulated intestinal microbiome comprising inoculating a liquid medium containing a nutritional energy source with a bacterial population comprising 5 to 200 bacterial species, wherein the liquid medium is sequentially inoculated with the bacterial species, and wherein the bacterial population digests the nutritional energy source, releasing at least 3 of butyrate, propionate, succinate, ethanol, acetate, lactate, and formate into the liquid medium.

53. The method of claim 52, wherein the bacterial species are sequentially added to the liquid medium based on their growth characteristics comprising slower growth characteristics or faster growth characteristics.

54. The method of claim 53, wherein the bacterial species with slower growth characteristics are added to the liquid medium before the bacterial species with faster growth characteristics.

55. The method of claim 52, wherein the stimulated intestinal microbiome comprises at least 16 bacterial species.

56. The method of claim 52, wherein the bacterial species comprises Bacteroides dorei, Bacteroides uniformis, Bacteroides thetaiotaomicron, Bacteroides vulgatus, Bifidobacterium longum, Blautia producta, Blautia sp., Bariatricus massiliensis, Clostridium innocuum, Dorea longicatena, Eubacterium hallii, Escherichia coli, Eubacterium rectale, Lactobacillus salivarus, Prevotella sp., or Roseburia faecis.

57. The method of claim 52, wherein the liquid medium comprises YFCA, Postgate's medium, Cooked meat broth, Peptone-yeast extract glucose broth, Thioglycollate broth, or SIEM medium.

58. The method of claim 52, wherein the liquid medium is YCFA.

59. The method of claim 52, wherein the nutritional energy source comprises monosaccharides, disaccharides, or oligosaccharides.

60. The method of claim 52, wherein the nutritional energy source comprises monosaccharides comprising fucose, rhamnose, hexose, or pentose.

61. An in vitro simulated intestinal microbiome comprising a liquid medium, a nutritional energy source and a bacterial population comprising 5 to 200 species of bacteria, wherein the bacterial population digests the nutritional energy source, releasing at least 3 metabolites selected from the group consisting of butyrate, propionate, succinate, ethanol, acetate, lactate, and formate into the liquid medium.

62. The in vitro simulated intestinal microbiome of claim 61, wherein the bacterial population comprises at most 16 species of bacteria.

63. The in vitro simulated intestinal microbiome of claim 61, wherein the bacterial population comprises at least 3 of (a)-(e):
(a) butyrate-producing bacteria optionally selected from the group consisting of Butyricicoccus, Eubacterium, Anaerostipes, Coprococcus, Butyrivibrio, Roseburia, Faecalibacterium, Bariatricus, Megasphaera, and Clostridium,
(b) propionate-producing bacteria optionally selected from the group consisting of Firmicutes, Verrucomicrobia, Bacteroidetes, Bacteroides, Blautia, Prevotella, and Eubacterium,
(c) acetate-producing bacteria optionally selected from the group consisting of Bacteroides, Bifidobacterium, Prevotella, Blautia, Selenomonas and Clostridium,
(d) lactate-producing bacteria optionally selected from the group consisting of Bifidobacterium, Bacteroides, Anaerostipes, Coprococcus, Clostridium, Collinsella, Roseburia and Lactobacillus, or
(e) formate-producing bacteria optionally selected from the group consisting of Escherischia, Ruminococcus and Bifidobacterium.

64. The in vitro simulated intestinal microbiome of claim 61, wherein the bacterial population comprises Bacteroides dorei, Bacteroides uniformis, Bacteroides thetaiotaomicron, Bacteroides vulgatus, Bifidobacterium longum, Blautia producta, Blautia sp., Bariatricus massiliensis, Clostridium innocuum, Dorea longicatena, Eubacterium hallii, Escherichia coli, Eubacterium rectale, Lactobacillus salivarus, Prevotella sp., or Roseburia faecis.

65. The in vitro simulated intestinal microbiome of claim 61, wherein the liquid medium comprises YFCA, Postgate's medium, Cooked meat broth, Peptone-yeast extract glucose broth, Thioglycollate broth, or SIEM medium.

66. A method of predicting in vivo metabolic activity or properties of a cell of interest comprising:
(i) providing a simulated intestinal microbiome comprising a liquid medium, a nutritional energy source, and a bacterial population comprising 5 to 200 bacterial species, wherein the simulated intestinal microbiome is prepared by sequentially inoculating the liquid medium with the bacterial species;
(ii) adding the cell of interest to the simulated intestinal microbiome; and
(iii) assessing the composition of the simulated intestinal microbiome.

67. The method of claim 66, wherein the bacterial species are sequentially added to the liquid medium based on their growth characteristics comprising slower growth characteristics or faster growth characteristics

68. The method of claim 66, further comprising stabilizing the simulated intestinal microbiome.

69. The method of claim 66, wherein said assessing comprises analyzing changes in the levels of metabolites produced by the simulated intestinal microbiome, bacterial abundance of the simulated intestinal microbiome, bacterial diversity of the simulated intestinal microbiome, or the growth rate of the cell of the interest.

70. The method of claim 66, wherein said assessing further comprises comparing the composition of the simulated intestinal microbiome with the cell of interest added to a composition of a simulated intestinal microbiome without a cell of interest added.

71. The method of claim 66, wherein said assessing is carried out 1 hour to 24 hours following the addition of the cell of interest to the simulated intestinal microbiome.
